(19) United States
(12) Patent Application Publication
(10) Pub. No.: US 2021/0153804 A1
(43) Pub. Date: May 27, 2021

(54) JOINT ANALYSIS PROBE

(71) Applicant: OULUN YLIOPISTO, Oulu (FI)

(72) Inventors: Jerome THEVENOT, Oulu (FI); Simo SAARAKKALA, Oulu (FI); Aleksei TIULPIN, Oulu (FI)

(21) Appl. No.: 17/047,314

(22) PCT Filed: Apr. 10, 2018
(86) PCT No.: PCT/FI2018/050257

§ 371 Date: Oct. 13, 2020

(51) Int. Cl. A61B 5/00; G01R 1/067; A61B 5/11; A61B 5/06

(52) U.S. Cl. CPC A61B 5/4528; A61B 5/061; A61B 5/11; G01R 1/06788

(57) ABSTRACT

Joint analysis probe (100). The probe (100) includes a frame (102). The probe (100) also includes a microphone (106) embedded into the frame (102) and configured to measure sounds (108) from a joint (162) of a subject (160) in a non-contact manner. The probe (100) also includes a raised rim (104) around the microphone (106) configured and positioned to be in contact with the subject (160) when the microphone (106) measures the sounds (108) from the joint (162), whereby the raised rim (104) attenuates an ambient noise (110) captured by the microphone (106).

### FIELD

The invention relates to a joint analysis probe.

### BACKGROUND

Joints can be affected by several conditions severely reducing their function, affecting mobility or even leading to working disability. Common pathologic conditions affecting the joints are osteoarthritis (OA) and other traumatic- or inflammatory-related diseases inducing deterioration of the joint. For more information, see the following documents:

Altman R D (1987). Overview of osteoarthritis. Am J Med. 83:65-69.

Cisternas M G, Murphy L, Sacks J J, Solomon D H, Pasta D J, Helmick C G (2016). Alternative Methods for Defining Osteoarthritis and the Impact on Estimating Prevalence in a US Population-Based Survey. Arthritis Care Res (Hoboken) 68(5):574-80.

OA is the most common musculoskeletal disorder and can occur in different joints (knee, hip, spine . . . ). This complex disorder has been long recognized as a major public health problem: in addition to the deterioration of the quality of individuals' life, it generates significant costs to society. First clinical symptoms of OA include pain during joint movement. Subsequently, when the disease gets worse, pain will occur also during rest and the function of joint will be significantly reduced. At the final stage, pain is intolerable making survival of routine daily activities highly difficult. The only treatment at this stage is joint replacement surgery, which is a major and relatively expensive operation requiring specialized healthcare. For more information, see the following documents:

Gellhorn A C, Katz J N, Suri P (2013). Osteoarthritis of the spine: the facet joints. Nat Rev Rheumatol. 9(4):216-24.

Pereira D, Peleteiro B, Araújo J, Branco J, Santos R A, Ramos E (2011). The effect of osteoarthritis definition on prevalence and incidence estimates: a systematic review. Osteoarthritis Cartilage 19 (11):1270-85.

OA involves multiple doctor appointments and expensive imaging examinations, often in specialized healthcare, due to its challenging diagnosis. While complete pharmaceutical cure of OA does not currently exist, the progression of the disease could be hindered by an early stage diagnosis.

Furthermore, the diagnostics of other joint conditions suffers comparable issues due to their subjective assessment and can also lead to OA if not treated properly.

For more information, see also the following documents:

Gunther K P, Sun Y (1999). Reliability of radiographic assessment in hip and knee osteoarthritis. Osteoarthritis and Cartilage 7:239-46.

Culvenor A G, Crossley K M (2016). Accelerated return to sport after anterior cruciate ligament injury: a risk factor for early knee osteoarthritis? Br J Sports Med. 50(5):260-1.

The primary drawbacks of current clinical diagnostics of joint conditions are:

1) time to get the final diagnosis can be long

2) both direct and indirect costs related to non-diagnosed joint conditions are high

3) false detection allows the joints to deteriorate further.

Eventually, a late diagnosis of OA reduces the available treatment options. Furthermore, for other joints conditions, a late diagnosis often causes the apparition of OA. From an economic point of view, alternative low-cost solutions available at the primary healthcare could replace some unnecessary and more expensive clinical examinations at the specialized healthcare related to joint diagnostics.

At the moment, the assessment of joint condition at the primary healthcare is performed using clinical (physical) examination, X-ray imaging and evaluation of symptoms (pain and limited joint movement). However, it is often difficult for a general practitioner to provide an objective and accurate diagnosis due to the insensitivity of clinical examination and X-ray imaging to tissue changes, especially in the case of soft tissues (ligaments, articular cartilage, menisci). Consequently, patients are quite often referred to specialized healthcare units where more comprehensive evaluation of the joint is possible, e.g., by using magnetic resonance imaging (MRI) or invasive arthroscopy.

The follow-up of post-traumatic or post-surgery patients is also a major concern involving not only physical rehabilitation, but also expensive “check-ups”. Currently, the patients follow specific rehabilitation programs by the help of a physiotherapist.

At the moment, accurate diagnosis of joint disorders (such as early OA) are challenging at the primary healthcare as they require multiple tests to reach decent specificity and sensitivity. To obtain further information, advanced techniques, i.e. expensive MRI or invasive arthroscopy, are typically performed at the specialized healthcare. While multiple studies have demonstrated the relevance of the modalities presented here, they are not used in clinical practice. So far, no device has been developed for clinical purpose with these sensors. Concerning the follow-up of patients post-surgery (or post-trauma), the options are limited to evaluate the improvements of the joint since the access to imaging modalities is restricted. Typically, the patients provide subjective self-reports of the improvements.

### BRIEF DESCRIPTION

The present invention seeks to provide an improved joint analysis probe.

According to an aspect of the present invention, there is provided a joint analysis probe as specified in claim 1.

The invention allows to perform a low-cost, comprehensive and efficient assessment of different joints. The invention could be used already at the primary healthcare as a supporting tool in joint diagnostics, offering also an opportunity of early OA screening. Furthermore, the developed technology will allow to diagnose other joints conditions as well, such as anterior cruciate ligament (ACL) injury for the knee or temporomandibular disorder for the jaw, chronic back pain, etc. Finally, the invention could help in the follow-up of patients by evaluating regularly the changes occurring in the concerned joint with time. Besides human beings, the invention may be used to diagnose and treat animals as well.

### LIST OF DRAWINGS

Example embodiments of the present invention are described below, by way of example only, with reference to the accompanying drawings, in which

FIG. 1 illustrates example embodiments of a joint analysis probe;

FIGS. 2A, 2B and 2C illustrate an example embodiment of a frame of the joint analysis probe;

FIG. 3 illustrates an example embodiment of a holder for sensors;

FIGS. 4, 5, 6, 7, 8 and 9 illustrate example embodiments of end parts;

FIGS. 10 and 11 illustrate further example embodiments of the joint analysis probe;

FIG. 12 illustrates various joints of a human being;

FIG. 13 illustrates an example embodiment of the joint analysis probe for a human being; and

FIG. 14 illustrates an example embodiment of the joint analysis probe for a horse.

### DESCRIPTION OF EMBODIMENTS

The following embodiments are only examples. Although the specification may refer to “an” embodiment in several locations, this does not necessarily mean that each such reference is to the same embodiment(s), or that the feature only applies to a single embodiment. Single features of different embodiments may also be combined to provide other embodiments. Furthermore, words “comprising” and “including” should be understood as not limiting the described embodiments to consist of only those features that have been mentioned and such embodiments may contain also features/structures that have not been specifically mentioned.

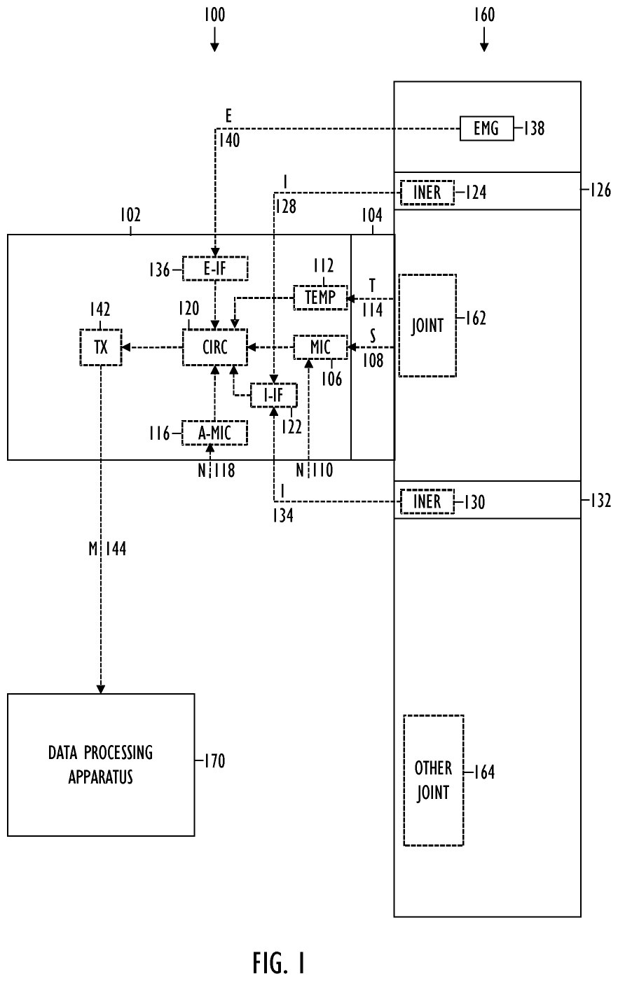

Let us first study FIG. 1 illustrating example embodiments of a joint analysis probe 100.

The probe 100 comprises a frame 102.

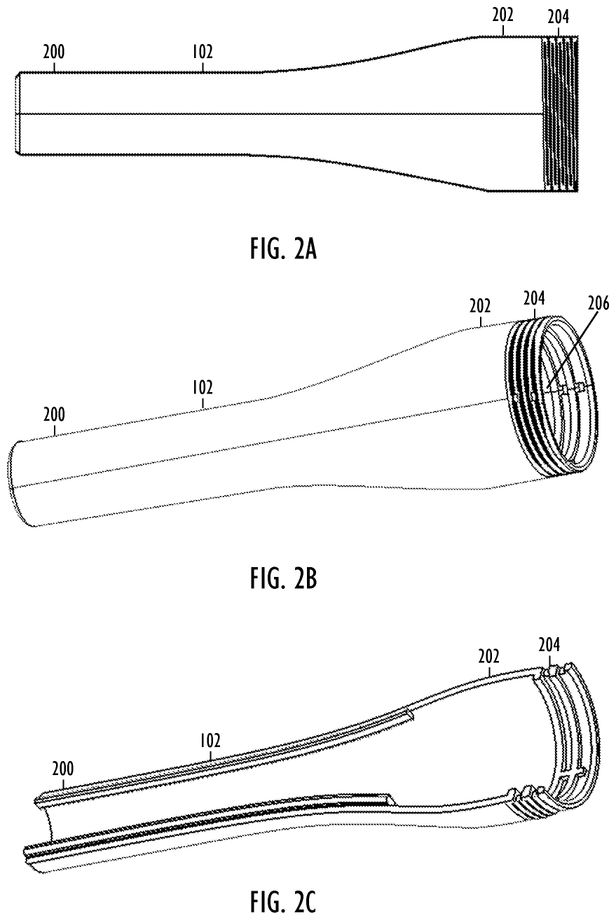

In an example embodiment illustrated in FIGS. 2A, 2B, 2C, 10, 11 and 13, the frame 102 is an elongated (rigid or semi-rigid) frame configured and dimensioned to be hand-held by a user.

In another example embodiment illustrated in FIG. 14, the frame 102 (which may or may not have the elongated shape) is configured and dimensioned to be attachable to a joint brace 1402.

The probe 100 also comprises a microphone 106 embedded into the frame 102 and configured to measure sounds 108 from a joint 162 of a subject 160 in a non-contact manner. The microphone 106 may be a non-contact microphone. The microphone 106 may be condenser microphone, for example, offering a wide frequency range and a high sensitivity.

The probe 100 also comprises a raised rim 104 around the microphone 106 configured and positioned to be in contact with the subject 160 when the microphone 106 measures the sounds 108 from the joint 162, whereby the raised rim 104 attenuates an ambient noise 110 captured by the microphone 106.

As shown in FIGS. 2A, 2B and 2C, the elongated frame 102 may be shaped like a flashlight having a narrow part 200 dimensioned to be held in hand, and a wider part 202 housing the microphone 106 and possibly also other sensor(s). The wide end 202 may have threads 204 to which an additional part may be attached.

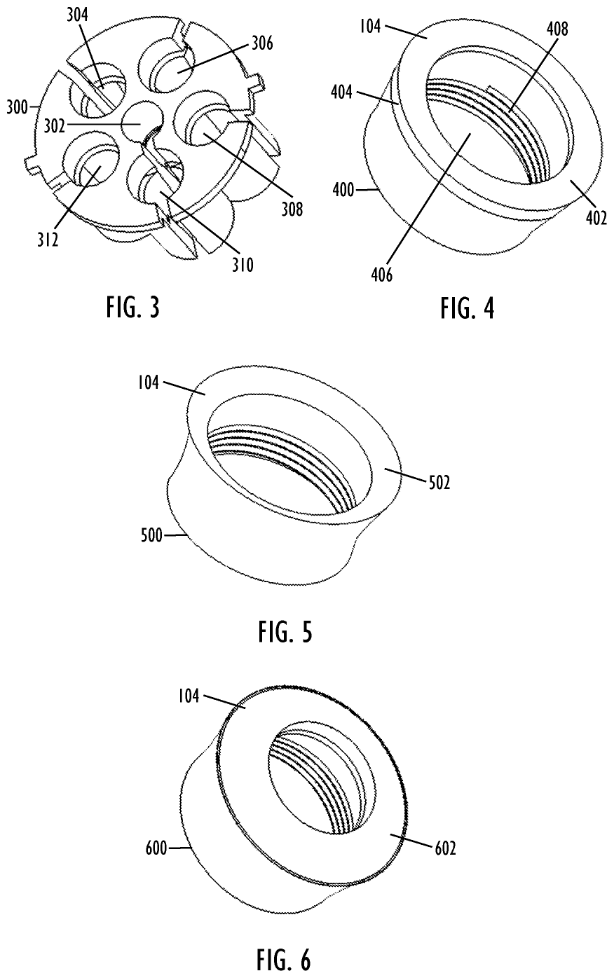

FIG. 3 illustrates a holder 300 for the sensors. The microphone 106 may be housed in a middle hollow 302. The holder 300 is attached to the wide end 202 of the elongated frame 102 (with snap-fitting or between the frame 102 and a further screwed-on part 400 shown in FIG. 4, for example).

Surrounding hollows 304, 306, 308, 310, 312 may house one or more temperature sensors 112.

In an example embodiment illustrated in FIGS. 1 and 3, the probe 100 further comprises one or more temperature sensors 112 next to the microphone 106 and configured to measure one or more temperatures 114 from the joint 162 in a non-contact manner.

Besides the microphone 106 and the temperature sensors 112, the probe 100 may comprise other sensors and/or sensor interfaces.

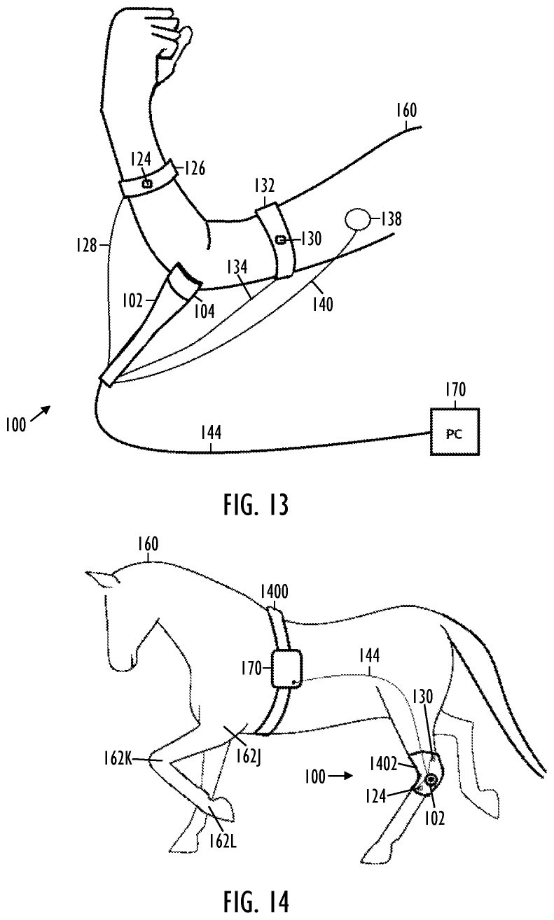

In an example embodiment illustrated in FIGS. 1 and 13, the probe 100 further comprises an inertial input interface 122 to couple with two optional inertial sensors 124, 130 attachable with straps 126, 132 close to the joint 162 and configured to measure inertial data 128, 134 during a movement of the joint 162.

The inertial sensor 124, 130 may comprise a six degrees of freedom inertial measurement unit Six degrees of freedom refers to the freedom of movement of a rigid body in three-dimensional space: change position as forward/backward (surge), up/down (heave), left/right (sway) translation in three perpendicular axes, combined with changes in orientation through rotation (pitch, yaw, and roll) about three perpendicular axes. The inertial sensor 124, 130 may detect a rate of acceleration using one or more accelerometers, and changes in rotational attributes (pitch, yaw and roll) using one or more gyroscopes.

In an example embodiment, illustrated in FIGS. 1 and 13 as well, the probe 100 further comprises an electromyography EMG input interface 136 to couple with optional EMG sensors 138 attachable close to the joint 162 and configured to measure EMG data 140 during a movement of the joint 162.

FIG. 4 illustrates an end part 400, which comprises counterpart threads 408 for the threads 204 of the elongated frame 102. The end part 400 comprises the rim 104, which surrounds the microphone 106 placed inside a hollow 406. The hollow 406 forms an empty space, which keeps the sensors from having a contact to the skin of the subject 160. The end part 400 may have a silicone part 404 for the contact against the skin. The purpose of the silicone part 404 is to reduce external noise and to be easy to wash. Besides silicone, other suitable flexible material may be used.

In FIG. 4, the shape 402 of the rim 104 against the skin of the subject 160 is flat.

As shown in FIGS. 2B and 4, the frame 102 comprising the end part 400 may have a hollow 206, 406, wherein the microphone 106 may be embedded.

FIG. 5 illustrates an alternative end part 500 having a concave shape 502 in the rim 104.

FIG. 6 illustrates an alternative end part 600 having a convex shape 602 in the rim 104.

The concave shape 502 may provide greater noise attenuation as a sealing between the rim 104 and the skin of the subject 160 may be better, but the convex shape 602 may in some situations be better due to the anatomical differences between different joints 162.

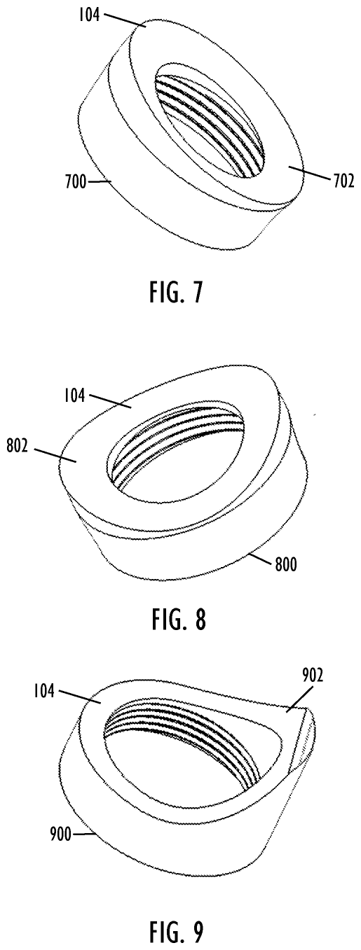

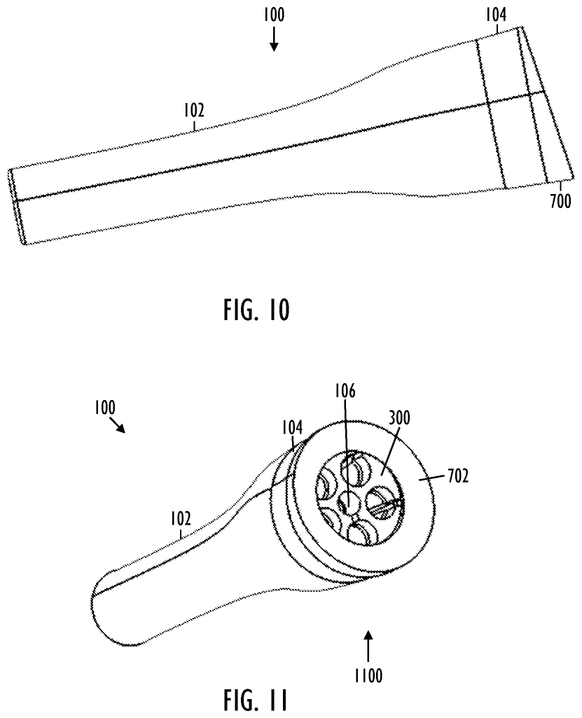

Besides flat, concave and convex shapes 402, 502, 602, other shapes are feasible: FIG. 7 illustrating an end part 700 with a straight slope 702, FIG. 8 illustrating an end part 800 with a partially concave slope 802, and FIG. 9 illustrating an end part 900 with a curved slope 902. FIGS. 10 and 11 illustrate the probe 100 with the end part 700.

In an example embodiment illustrated in FIG. 11, the microphone 106 is embedded into a cup-shaped earmuff like structure 1100 configured and dimensioned to partly surround the joint 162 in order to attenuate the ambient noise 110 captured by the microphone 106.

In an example embodiment, illustrated in FIGS. 4, 5, 6, 7, 8, 9, 10 and 11, a part 400/500/600/700/800/900 comprising the raised rim 104 is removably attachable with the (elongated) frame 102, and the part 400/500/600/700/800/900 belongs to a set of parts 400, 500, 600, 700, 800, 900, and each part 400, 500, 600, 700, 800, 900 of the set is configured and dimensioned to measure a different joint 162, 164 of the subject 160.

Such configuration and dimensioning may be made with the described shapes 402, 502, 602, 702, 802, 902. Also, sizing may be made for different age groups, infants, children and adult, or for different sexes, men and women, for example. Configuration and dimensioning may also be made for different types of subjects 160, such as for human beings and/or for different animal species.

In an example embodiment, the probe 100 is configured and dimensioned to fit physiological properties of a human being 160.

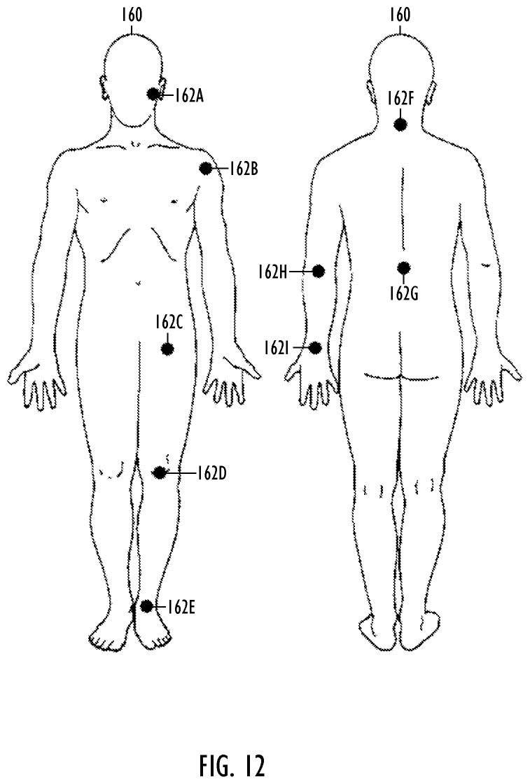

FIG. 12 illustrates various joints (such as temporomandibular, neck, shoulder, elbow, lower back, wrist, hip, knee and ankle) 162A-162I of the human being 160, which may necessitate different configurations and dimensions (whereby the tip 500, 600, 700, 800, 900 of the probe 100 may be changed to fit the joint studied). FIG. 13 illustrates the examination of the elbow joint 162H with the probe 100.

In an example embodiment, the probe 100 is configured and dimensioned to fit physiological properties of an animal, including one or more of the following: a horse, a camel, a dog.

FIG. 14 illustrates the examination of the knee of the horse 160 with the probe 100. Typical joints of the horse 160 for the examination are elbow 162J, knee 162K and fetlock 162L. The probe 100 with its parts 100, 124, 130 may be attachable to a joint brace such as a commercially available hock brace 1402, or, depending on the horse joint studied, another commercially available joint brace such as a fetlock brace.

In an example embodiment illustrated in FIG. 1, the probe 100 further comprises an additional microphone 116 configured to measure the ambient noise 118, and an electronic circuit 120 configured to generate a waveform that is a negative of the ambient noise 118 and mix the waveform with the sounds 108 measured from the joint 162 in order to cancel the ambient noise 110. With this example embodiment, the attenuation of the ambient noise 110 achieved with the raised rim 104 is further enhanced with the active noise cancellation.

In an example embodiment illustrated in FIGS. 1, 13 and 14, the probe 100 further comprises a transmitter 142 (or a transceiver for two-way communication) configured to communicate the measurements 144 to an external data processing apparatus 170. As shown in FIGS. 13 and 14, the external data processing apparatus 170 may be local, and even strapped to the subject 160 with a strap 1400. But, as well, the external data processing apparatus 170 may be remote. The data communication 144 between the probe 100 and the external data processing apparatus 170 may be implemented with wired or wireless communication means.

Another patent application by the applicant, PCT/FI2017/050760, describes a somewhat similar distributed configuration, and is incorporated herein by reference in all jurisdictions where applicable.

The probe 100 has been designed to be totally non-invasive and painless to use. As explained, the probe 100 may include up to six non-contact internal sensors: five thermal sensors 112 and a microphone 106 at its extremity (below the tip). Different tips of the probe 100 are available, each of them specific to the shape of the joint 162 studied. The tips 500, 600, 700, 800, 900 may be attached to the body 102 of the probe 100, and they all contain a material to reduce external noise and a silicon-based extremity (easy to wash) to be in contact with the skin. Two or more kinetic sensors 124, 130 may be externally connected to the probe 100, each of them embedded on a different strap 126, 132 to be positioned from each side of the studied joint 162 and are synchronized with all the sensors 106, 112. Multiple external electrodes 138 may be connected to the probe 100 to perform electromyography (EMG) simultaneously.

The data acquisition is performed by acquiring signals from the studied joint 162 using the following protocol. The kinetic sensors 124, 130 are placed on each side of the studied joint 162 by the help of the straps 126, 132 to measure velocity and angles of rotation (optional. The EMG electrodes 138 are placed on the muscles of interest to collect information of their activity (optional). The user chooses the tip 500, 600, 700, 800, 900 of the probe 100 according to the examined joint 162 and attaches it on the probe 100. The user keeps the probe 100 on the surface of the skin of the studied joint 162 while the patient 160 moves the joint 162 (e.g. flexion-extension, sit-to-stand, bending).

The basis of choosing these modalities are as follows. Acoustic modality assesses the friction of the cartilage at the joint 162, which is an indicator of articular cartilage degeneration and wear. Thermal modality assesses the inflammation of the joint 162. Kinetic modality provides information on joint 162 malalignment, angular velocity and bending. Electromyography provides information on muscular activity.

The multi-modal analysis is performed as follows. After the signal acquisition, the automatic data analysis is performed and relevant features for each modality are extracted (e.g. differences in temperature, amount of acoustic emissions above a given threshold, angles of motion, muscular activity, etc.). In addition to the signal data, other anthropometric variables such as age and body mass index of the subject 160 may be incorporated to the algorithm evaluating the joint 162 condition. The final diagnostics is performed as an overall assessment of all the signals collected.

The combination of multiple modalities together results in the overall assessment of the joint 160, and diagnostic of disorders/follow-up of joint condition.

As a final result, a report providing all the characteristics obtained from the signals is given, with overall estimation of the joint 162 condition and potential diagnostic if relevant.

For more information, see the following documents:

Zhang W, Doherty M, et al (2009). EULAR evidence-based recommendations for the diagnosis of hand osteoarthritis: report of a task force of ESCISIT. Annals of the Rheumatic Diseases 68:8-17.

Mascaro B I, Prior J, Shark L K, Selfe J, Cole P, Goodacre J (2009). Exploratory study of a non-invasive method based on acoustic emission for assessing the dynamic integrity of knee joints. Med Eng Phys. 31(8): 1013-22.

Bassiouni H M (2012). Phonoarthrography: A New Technique for Recording Joint Sounds, Osteoarthritis—Diagnosis, Treatment and Surgery, Prof. Qian Chen (Ed.), InTech, DOI: 10.5772/25981.

Ammer K (2012). Temperature of the human knee—a review. Thermology international 22(4): 137-51.

Chang A, Hochberg M, Song J, Dunlop D, et al. (2010). Frequency of varus and valgus thrust and factors associated with thrust presence in persons with or at higher risk of developing knee osteoarthritis. Arthritis Rheum. 62(5):1403-11.

In an example embodiment, the external data processing apparatus 170 is a computing device. It may be portable, mobile or stationary. A non-limiting list of example embodiments of the external data processing apparatus 170 comprises but is not limited to: a computer, a portable computer, a laptop, a mobile phone, a smartphone, a tablet computer, a smartwatch, smartglasses, or any other portable/mobile/stationary computing device. The external data processing apparatus 170 may output data related to the measurements with a user interface. The external data processing apparatus 170 may comprise a sound card for processing the measured sounds 108.

In an example embodiment, the external data processing apparatus 170 is a computing server. It may be implemented with any applicable technology. It may include one or more centralized computing apparatuses, or it may include more than one distributed computing apparatuses. It may be implemented with client-server technology, or in a cloud computing environment, or with another technology applicable to the external data processing apparatus 170 capable of communicating 144 with the probe 100.

In an example embodiment, the probe 100 may be an independent integrated apparatus comprising also the external data processing apparatus 170.

In an example embodiment, the probe 100 is sold as a product in itself, or the use of the probe 100 is marketed as a service per use of the device (the analysis of the signal is performed remotely and the results are sent back to the customer).

The primary healthcare (both public and private) may use the probe 100. At the public healthcare the probe 100 and the service may be used already in the health centres and the test itself may be supervised by a nurse or other trained person. At the private healthcare, big health clinics as well as private physiotherapists will be the first targeted customers. The probe 100 will provide a complementary source of information to the practitioners for the diagnosis of joint 162 disorders. The easy access to this information already at the primary healthcare will prevent extra expenses related to unnecessary advanced examinations and doctor appointments at the specialized healthcare.

Rehabilitation centres and physiotherapists may use the probe 100 to follow the evolution of the joints 162 during follow-up

Sports centres may use the probe 100 to assess the quality of the joints 162 of athletes.

Companies developing orthopaedic devices may use the probe 100 to validate the design of their product from follow-up populations.

Veterinary clinics may use the probe 100 on animals.

In an example embodiment, the coupling 144 is wired, employing suitable standard or proprietary bus and protocol. In an example embodiment, the coupling 144 is wireless employing a radio transmitter 142. In an example embodiment, the radio transmitter 142 is a part of a radio transceiver. In an example embodiment, the radio transceiver 142 comprises a cellular radio transceiver (communicating with technologies such as GSM, GPRS, EGPRS, WCDMA, UMTS, 3GPP, IMT, LTE, LTE-A, etc.) and/or a non-cellular radio transceiver (communicating with short-range technologies such as Bluetooth, Bluetooth Low Energy, Wi-Fi, WLAN, etc.). With the cellular radio transceiver, the probe 100 and the external data processing apparatus 170 may be distributed so that they are located in the same town, in different towns, or even in different continents. With the non-cellular radio transceiver, the probe 100 and the external data processing apparatus 170 need to be near each other, in the same room or in the same building, for example, except if there is a communication network in between (such as a wireless access point connected to the Internet), then the distribution degree may be the same as with the cellular radio transceiver. Note that the use of the cellular radio transceiver may necessitate the use of a subscriber identity module (SIM), and, consequently, the probe 100 comprises a SIM card in a card reader, or a virtual (or software) SIM.

Note that the probe 100 may comprise other parts as well, which have not been described, but are naturally there: a power source (such as battery, which may be rechargeable) to feed electric energy to the sensors (and possibly for the condenser microphone 106) and also an interface, which collects the measurement data from the sensors to communicate the measurement data to the external data processing apparatus 170. The measurement data may be raw data from the sensors, or it may be pre-processed in the probe 100 before communicated to the external data processing apparatus 170.

It will be obvious to a person skilled in the art that, as technology advances, the inventive concept can be implemented in various ways. The invention and its embodiments are not limited to the example embodiments described above but may vary within the scope of the claims.

1. A joint analysis probe comprising:
a frame;
a microphone embedded into the frame and configured to measure sounds from a joint of a subject in a non-contact manner; and
a raised rim around the microphone configured and positioned to be in contact with the subject when the microphone measures the sounds from the joint, whereby the raised rim attenuates an ambient noise captured by the microphone.

2. The joint analysis probe of claim 1, wherein the frame is an elongated frame configured and dimensioned to be hand-held by a user.

3. The joint analysis probe of claim 1, wherein the frame is configured and dimensioned to be attachable to a joint brace.

4. The joint analysis probe of claim 1, wherein the microphone is embedded into an end of the frame.

5. The joint analysis probe of claim 1, wherein the microphone is embedded into a hollow of the frame.

6. The joint analysis probe of claim 1, wherein the microphone is embedded into a cup-shaped earmuff like structure configured and dimensioned to partly surround the joint in order to attenuate the ambient noise captured by the microphone.

7. The joint analysis probe of claim 1, wherein a part comprising the raised rim is removably attachable with the frame, and the part belongs to a set of parts, and each part of the set is configured and dimensioned to measure a different joint of the subject.

8. The joint analysis probe of claim 1, further comprising an additional microphone configured to measure the ambient noise, and an electronic circuit configured to generate a waveform that is a negative of the ambient noise and mix the waveform with the sounds measured from the joint in order to cancel the ambient noise.

9. The joint analysis probe of claim 1, further comprising one or more temperature sensors next to the microphone and configured to measure one or more temperatures from the joint in a non-contact manner.

10. The joint analysis probe of claim 1, further comprising an inertial input interface to couple with two optional inertial sensors attachable with straps close to the joint and configured to measure inertial data during a movement of the joint.

11. The joint analysis probe of claim 1, further comprising an electromyography EMG input interface to couple with optional EMG sensors attachable close to the joint and configured to measure EMG data during a movement of the joint.

12. The joint analysis probe of claim 1, further comprising a transmitter configured to communicate the measurements to an external data processing apparatus.

13. The joint analysis probe of claim 1, wherein the joint analysis probe is configured and dimensioned to fit physiological properties of a human being.

14. The joint analysis probe of claim 1, wherein the joint analysis probe is configured and dimensioned to fit physiological properties of an animal, including one or more of the following: a horse, a camel, a dog.
